(19) United States
(12) Patent Application Publication
(10) Pub. No.: US 2006/0269596 A1
(43) Pub. Date: Nov. 30, 2006

(54) Controlled release compositions comprising an acylanilide

(76) Inventors: Gary Liversidge, West Chester, PA (US); Scott Jenkins, Downingtown, PA (US)

Correspondence Address: SYNNESTVEDT & LECHNER LLP;ELAN CORPORATION PLC, 1101 MARKET STREET, SUITE 2600, PHILADELPHIA, PA, 19107-2950, US

(21) Appl. No.: 11/329,893

(22) Filed: Jan. 11, 2006

Related U.S. Application Data
(60) Provisional application No. 60/643,725, filed on Jan. 12, 2005.

(51) Int. Cl. A61K 9/64; A61K 9/26

(52) U.S. Cl. 424/456; 424/470

(57) ABSTRACT

The invention relates to a controlled release composition comprising acylanilide, and preferably bicalutamide, for use, in particular, in combination therapy with a luteinizing hormone-releasing hormone (LHRH) analogue for the treatment of stage D₂ metastatic carcinoma of the prostate. The controlled release composition comprises an immediate release component and a modified release component or formulation. The immediate release component comprises a first population of bicalutamide. The modified release formulation preferably comprises a second population of acylanilide bicalutamide, and a controlled release constituent. The controlled release formulation is preferably in the form of an erodable formulation, a diffusion controlled formulation or an osmotic controlled formulation. The combination of the immediate release and modified release components in operation deliver the active ingredient in a pulsed or bi-modal manner.

### CROSS REFERENCE TO RELATED APPLICATION

The present application claims the priority benefit under 35 U.S.C. §1.119(e) of the Jan. 12, 2005 filing date of U.S. provisional Application No. 60/643,725.

### FIELD OF INVENTION

The present invention relates generally to the treatment of cancer and, in particular, to combination therapies for the treatment of carcinoma of the prostate. More specifically, the present invention comprises controlled release compositions consisting of an acylanilide, and preferably bicalutamide, for use, preferably, in combination therapy with a luteinizing hormone-releasing hormone (LHRH) analogue for the treatment of stage D₂ metastatic carcinoma of the prostate.

A preferred embodiment of the present invention relates to a nanoparticulate composition comprising an acylanilide such as bicalutamide formulated in a number of controlled release delivery systems, resulting in an increased bioavailability of the otherwise poorly water soluble drug that is released over a sustained period of time.

### BACKGROUND OF THE INVENTION

A. Background Regarding Nanoparticulate Compositions

Nanoparticulate compositions, first described in U.S. Pat. No. 5,145,684 (“the '684 patent”), are particles consisting of a poorly soluble therapeutic or diagnostic agent having adsorbed onto the surface thereof a non-crosslinked surface stabilizer. The '684 patent does not describe nanoparticulate compositions of an acylanilide.

Methods of making nanoparticulate compositions are described in, for example, U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and United States Pat. No. 5,510,118 for “Process of Preparing Therapeutic Compositions Containing Nanoparticles.”

Nanoparticulate compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(-)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive Nanoparticulate Compositions Having Cationic Surface Stabilizers;” U.S. Pat. No. 6,431,478 for “Small Scale Mill;” and U.S. Pat. No. 6,432,381 for “Methods for Targeting Drug Delivery to the Upper and/or Lower Gastrointestinal Tract,” all of which are specifically incorporated by reference. In addition, U.S. Patent Application No. 20020012675 A1, published on Jan. 31, 2002, for “Controlled Release Nanoparticulate Compositions,” describes nanoparticulate compositions, and is specifically incorporated by reference.

Amorphous small particle compositions are described, for example, in U.S. Pat. No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.”

B. Background Regarding Bicalutamide (CASODEX®)

Compositions of the invention comprise an acylanilide, and most preferably, bicalutamide. Bicalutamide is offered under the registered trademark CASODEX® by AstraZeneca Pharmaceuticals, LP, of Wilmington, Del. The Physicians Desk Reference, 58^{th} Ed., pp. 3, 306 (2004).

Bicalutamide, also known as propanamide, N-[4-cyano-3-(trifluoromethyl)phenyl]-3-[4-fluorophenyl) sulfonyl]-2-hydroxy-2-methyl-, (+ −) is a non-steroidal anti-androgen with no other endocrine activity. Bicalutamide is a fine white to off-white powder offered as a tablet. Bicalutamide is practically insoluble in water at 37 C (5 mg per 1000 mL). CASODEX® is a racemate with its anti-androgenic activity being almost exclusively exhibited by the R-enantiomer of bicalutamide; the S-enantiomer is essentially inactive. The Physicians Desk Reference, 58^{th} Ed., p. 658 (2004).

Amide derivatives such as bicalutamide are described in, for example, U.S. Pat. No. 4,636,505 to Tucker. The Tucker patent refers to a class of acylanilides and is incorporated herein by reference.

Since bicalutamide is a non-steroidal anti-androgen, it competitively inhibits the action of androgens by binding to cytosol androgen receptors in the target tissue. Prostatic carcinoma is known to be androgen sensitive and responds to treatment that counteracts the effect of androgen and/or removes the source of androgen.

When bicalutamide is combined with luteinizing hormone-releasing hormone (LHRH) analogue therapy, the suppression of serum testosterone inducted by the LHRH analogue is not affected. However, in clinical trials with bicalutamide as a single agent for prostate cancer, rises in serum testosterone and estradiol have been noted. Bicalutamide is well-absorbed following oral administration, although the absolute bioavailability is unknown. Co-administration of bicalutamide with food has no clinically significant effect on rate or extent of absorption. Bicalutamide is highly protein-bound (96%). Bicalutamide undergoes stereo-specific metabolism. The S (inactive) isomer is metabolized primarily by glucuronidation. The R (active) isomer also undergoes glucuronidation but is predominantly oxidized to an inactive metabolite followed by glucuronidation. Both the parent and metabolite glucuronides are eliminated in the urine and feces. The S-enantiomer is rapidly cleared relative to the R-enantiomer, with the R-enantiomer accounting for about 99% of total steady-state plasma levels. The Physicians Desk Reference, 58^{th} Ed., pp. 3, 306 (2004).

Because conventional bicalutamide tablets are practically insoluble in water at 37 C (5 mg per 1000 mL), bicalutamide tablets have limited bioavailability, which limits the therapeutic outcome for all treatments requiring bicalutamide. There is a need in the art for bicalutamide formulations which overcome these and other problems associated with bicalutamide.

### SUMMARY OF THE INVENTION

It is an object of the present invention to provide a controlled release composition containing acylanilide, and preferably bicalutamide, which in operation produces a plasma profile substantially similar to the plasma profile produced by the administration of two or more immediate release (LR) dosage forms given sequentially.

It is a further object of the invention to provide a controlled release composition, which in operation delivers acylanilide, and preferably bicalutamide, in a pulsatile manner.

Another object of the invention is to provide a controlled release composition which substantially mimics the pharmacological and therapeutic effects produced by the administration of two or more immediate release dosage forms given sequentially.

Another object of the present invention is to provide a controlled release composition which substantially reduces or eliminates the development of patient tolerance to the acylanilide, and preferably bicalutamide, of the composition.

Another object of the invention is to provide a controlled release composition in which a first portion of the acylanilide, and preferably bicalutamide, is released immediately upon administration and a second portion of the acylanilide, and preferably bicalutamide, is released rapidly after an initial delay period in a bimodal manner.

Another object of the present invention is to formulate the dosage in the form of erodable formulations, diffusion controlled formulations or osmotic controlled formulations.

Another object of the invention is to provide a controlled release composition capable of releasing the acylanilide, and preferably bicalutamide, in a bimodal or multi-modal manner in which a first portion of the active is released either immediately or after a delay time to provide a pulse of drug release, and one or more additional portions of the acylanilide, and preferably bicalutamide, is released, each after a respective lag time, to provide additional pulses of drug release during a period of up to twenty-four hours.

Another object of the invention is to provide solid oral dosage forms comprising a controlled release composition of the present invention, comprising bicalutamide.

Other objects of the invention include provision of a once daily dosage form of bicalutamide which, in operation, produces a plasma profile substantially similar to the plasma profile produced by the conventional administration of two immediate release dosage forms given sequentially and a method for treatment, in particular, in combination therapy with a luteinizing hormone-release hormone (LHRH) analogue for the treatment of stage D₂ metastatic carcinoma of the prostate based on the administration of such a dosage form.

The above objects are realized by a controlled release composition having a first component comprising a first population of acylanilide, and preferably bicalutamide, particles and a second component or formulation comprising a second population of acylanilide, and preferably bicalutamide, particles. The ingredient-containing particles of the second component further comprises a modified release constituent comprising a release coating or release matrix material, or both. Following oral delivery, the composition in operation delivers the acylanilide, and preferably bicalutamide, in a pulsatile manner.

The present invention utilizes controlled release delivery of acylanilide, and preferably bicalutamide, from a solid oral dosage formulation to allow dosage less frequently than before, and preferably once-a-day administration, increasing patient convenience and compliance. The mechanism of controlled release would preferably utilize, but not be limited to, erodable formulations, diffusion controlled formulations and osmotic controlled formulations. A portion of the total dose may be released immediately to allow for rapid onset of effect. The invention would be useful in improving compliance and, therefore, therapeutic outcome for all treatments requiring bicalutamide, including, in particular, in combination therapy with a luteinizing hormone-release hormone (LHRH) analogue for the treatment of stage D₂ metastatic carcinoma of the prostate. This approach would replace conventional bicalutamide tablets and solution, which are administered twice a day for such combination therapy.

The present invention also relates to a controlled modified release composition for the controlled release of acylanilide, and preferably bicalutamide. In particular, the present invention relates to a controlled release composition that in operation delivers acylanilide, and preferably bicalutamide, in a pulsatile manner, preferably during a period of up to twenty-four hours. The present invention further relates to solid oral dosage forms containing a controlled release composition.

Preferred controlled release formulations are erodable formulations, diffusion controlled formulations and osmotic controlled formulations. According to the invention, a portion of the total dose may be released immediately to allow for rapid onset of effect, with the remaining portion of the total dose released over an extended time period. The invention would be useful in improving compliance and, therefore, therapeutic outcome for all treatments requiring bicalutamide, including, in particular, in combination therapy with a luteinizing hormone-release hormone (LHRH) analogue for the treatment of stage D₂ metastatic carcinoma of the prostate.

The present invention preferably utilizes nanoparticulate compositions comprising an acylanalide, and preferably bicalutamide. The compositions comprise nanoparticulate bicalutamide particles, and at least one surface stabilizer adsorbed on the surface of the bicalutamide particles. The nanoparticulate bicalutamide particles have an effective average particle size of less than about 2000 nm.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

Controlled release compositions similar to those disclosed herein are disclosed and claimed in the U.S. Pat. Nos. 6,228,398 and 6,730,325 to Devane et al., both of which are incorporated by reference herein.

U.S. Provisional Application No. 60/638826, filed Dec. 22, 2004, entitled “Nanoparticulate Bicalutamide Formulations,” which describes the nanoparticulate bicalutamide formulations and methods of making bicalutamide nanoparticles, preferably employed in the controlled release compositions of the present invention, is also specifically incorporated by reference herein.

In a preferred embodiment of a controlled release composition according to the invention, the first component includes an immediate release constituent.

In the second component, the modified release coating applied to the second population or presence of a modified release matrix in the second population of acylanilide, and preferably bicalutamide, particles causes a lag time between the release of active from the first and second populations of active bicalutamide-containing, particles The duration of the lag time may be varied by altering the type and/or amount of the modified release coating and/or altering the type and/or amount of modified release matrix material utilized in the second or subsequent component or formulation. Preferred types of formulations for use in varying the lag time are erodable formulations, diffusion controlled formulations and osmotic controlled formulations. Thus, the duration of the lag time can be designed to mimic a desired plasma profile.

Erodable Formulations

The subsequent formulations can be in the form of erodable formulations in which the active ingredients and modified release constituent consisting of at least one of modified release coatings and modified release matrix materials would dissolve in water, over time losing their structural integrity. One manner in which this could occur would be that the active ingredients and modified release coatings and/or matrix materials would dissolve after human ingestion over a controlled period of time.

Diffusion Controlled Formulations

The subsequent formulations can be in the form of diffusion controlled formulations which would allow the gradual spread of the subsequent population of particles to scatter or spread out in a liquid medium, are referenced, for example, in U.S. Pat. No. 6,586,006 to Roser et al., which is incorporated by reference herein.

Osmotic Controlled Formulations

Controlled release of the subsequent formulations could be controlled by osmosis. U.S. Pat. No. 6,110,498 to Rudnic et al. for an “osmotic drug delivery system” discloses a system which dispenses a therapeutic agent having limited water solubility in solubilized form. The delivery system comprises a core that is free of swellable polymers and comprises nonswelling solubilizing agents and wicking agents. The solubilized therapeutic agent is delivered through a passageway in the semipermeable coating of the tablet.

U.S. Pat. No. 5,814,979 B2 also to Rudnic et al. describes an osmotic pharmaceutical delivery system comprising (a) a semi-permeable wall that maintains its integrity during pharmaceutical delivery and which has at least one passage therethrough; (b) a single, homogeneous composition within said wall, which composition consists essentially of (I) a pharmaceutically active agent, (ii) at least one non-swelling solubilizing agent which enhances the solubility of the pharmaceutically active agent; (iii) at least one non-swelling osmotic agent and (iv) a non-swelling wicking agent dispersed throughout the composition which enhances the surface area contact of the pharmaceutical agent with the incoming aqueous fluid. Both of these patents to Rudnic et al. are incorporated by reference herein.

Plasma Profile

The plasma profile associated with the administration of a drug compound may be described as a “pulsatile profile” in which pulses of high drug concentration, interspersed with low concentration troughs, are observed. A pulsatile profile containing two peaks may be described as “bimodal.” Similarly, a composition or a dosage form which produces such a profile upon administration may be said to exhibit “pulsed release” of the drug.

Conventional frequent dosage regimes in which an immediate release (IR) dosage form is administered at periodic intervals typically gives rise to a pulsatile plasma profile. In this case, a peak in the plasma drug concentration is observed after administration of each IR dose with troughs (regions of low drug concentration) developing between consecutive administration time points. Such dosage regimes (and their resultant pulsatile plasma profiles) have particular pharmacological and therapeutic effects associated with them. For example, the wash-out period provided by the fall off of the plasma concentration of the drug between peaks has been thought to be a contributing factor in reducing or preventing patient tolerance to various types of drugs.

Because the plasma profile produced by the controlled release composition upon administration is substantially similar to the plasma profile produced by the administration of two or more IR dosage forms given sequentially, the controlled release composition of the present invention is particularly useful for administering bicalutamide for which patient tolerance may be problematical. The controlled release compositions of the present invention are, therefore, advantageous for reducing or minimizing the development of patient tolerance to the active ingredient in the composition.

In the present invention, the active composition is acylanilide active, and preferably bicalutamide, and the composition in operation delivers the bicalutamide in a bimodal or pulsed manner. Such a composition in operation produces a plasma profile which substantially mimics that obtained by the sequential administration of two IR doses as, for instance, in a standard bicalutamide treatment regime.

The present invention also provides solid oral dosage forms comprising a composition according to the invention. The present invention further provides a method of treating a patient, in particular, in combination therapy with a luteinizing hormone-release hormone (LHRH) analogue for the treatment of stage D₂ metastatic carcinoma of the prostate utilizing bicalutamide, comprising the administration of a single daily therapeutically effective amount of a composition or solid oral dosage form according to the invention to provide a pulsed or bimodal administration of the bicalutamide. Advantages of the present invention include reducing the dosing frequency required by conventional multiple IR dosage regimes while still maintaining the benefits derived from a pulsatile plasma profile. This reduced dosing frequency is advantageous in terms of patient compliance to have a formulation which may be administered at reduced frequency. The reduction in dosage frequency made possible by utilizing the present invention would contribute to reducing health care costs by reducing the amount of time spent by health care workers on the administration of drugs.

Definitions

The term “particulate” as used herein refers to a state of matter which is characterized by the presence of discrete particles (including, and preferably nanoparticles), pellets, beads or granules irrespective of their size, shape or morphology. The term “multiparticulate” as used herein means a plurality of discrete, or aggregated, particles, pellets, beads, granules or mixture thereof irrespective of their size, shape or morphology.

The term “controlled release” as used herein in relation to the compositions according to the invention or used in any other context means release of acylanilide, and preferably bicalutamide, over time and is taken to encompass sustained release and delayed release.

The term “time delay” as used herein refers to the duration of time between administration of the composition and the release of the acylanilide, and preferably bicalutamide, from a particular component.

The term “lag time” as used herein refers to the time between delivery of bicalutamide from one component and the subsequent delivery of the drug from another component.

The active ingredient in each component consists of acylanilide, and preferably bicalutamide, although a second active ingredient having utility in combination therapy with a luteinizing hormone-release hormone (LHRH) analogue for the treatment of stage D₂ metastatic carcinoma of the prostate may be desirable for combination therapies.

Indeed, two or more active ingredients may be incorporated into the same component when the active ingredients are compatible with each other. The acylanilide, and preferably bicalutamide, present in one component of the composition may be accompanied by, for example, an enhancer compound or a sensitizer compound in another component of the composition, in order to modify the bioavailability or therapeutic effect of the active ingredient.

Additives

The acylanilide, and preferably bicalutamide, present in the first and second or subsequent components of the composition may be accompanied by, for example, an enhancer compound or a sensitizer compound in order to modifiy the bioavailability or the therapeutic effect of the bicalutamide.

As used herein, the term “enhancer” refers to a compound which is capable of enhancing the absorption and/or bioavailability of an active ingredient by promoting net transport across the gastro-intestinal tract in an animal, such as a human. Enhancers include but are not limited to medium chain fatty acids; salts, esters, ethers and derivatives thereof, including glycerides and triglycerides; non-ionic surfactants such as those that can be prepared by reacting ethylene oxide with a fatty acid, a fatty alcohol, an alkylphenol or a sorbitan or glycerol fatty acid ester; cytochrome P450 inhibitors, P-glycoprotein inhibitors and the like; and mixtures of two or more of these agents.

Proportion of Bicalutamide and Additives

The proportion of the acylanilide, and preferably bicalutamide, contained in each component may be the same or different depending on the desired dosing regime. The acylanilide, and preferably bicalutamide, is present in the first component and in the second component in any amount sufficient to elicit a therapeutic response. The bicalutamide when applicable, may be present either in the form of one substantially optically pure enantiomer or as a mixture, racemic or otherwise, of enantiomers. The bicalutamide may be present in a composition in an amount of from 0.1-500 mg, and is present preferably in the amount of from 1-50 mg. Bicalutamide is present in the first component preferably in an amount of from 2.5-30 mg. The bicalutamide is present in the subsequent components in an amount within a similar range to that described for the first component.

Time Release Profiles

The time release characteristics for the release of the acylanilide, and preferably bicalutamide, from each of the components may be varied by modifying the composition of each component, including modifying any of the excipients or coatings which may be present. In particular, the release of acylanilide, and preferably bicalutamide, may be controlled by changing the modified release constituent, including the amount of the modified release coating on the particles, if such a coating is present. As noted above, the time release profiles may be controlled by making the subsequent components or formulations in the form of erodable formulations, diffusion controlled formulations or osmotic controlled formulations. If more than one modified release constituent is present, the modified release coating for each of the subsequent components may be the same or different. Similarly, when modified release is facilitated by the inclusion of a modified release matrix material, release of the active ingredient may be controlled by the ingredient and amount of modified release matrix material utilized. The modified release coating may be present, in each component, in any amount that is sufficient to yield the desired delay time for each particular component. The modified release coating may be preset, in each component, in any amount that is sufficient to yield the desired time lag between components.

The lag time or delay time for the release of the bicalutamide from each component may also be varied by modifying each of the components, including modifying any excipients and coatings which may be present. For example, the first component may be an immediate release component wherein the bicalutamide is released substantially immediately upon administration. Alternatively, the first component may be, for example, a time-delayed immediate release component in which the bicalutamide is released after a time delay. The second component may be, for example, a time-delayed immediate release component as just described or, alternatively, a time-delayed sustained release or extended release component in which the bicalutamide is released in a controlled fashion for up to twenty-four hours.

Plasma Concentration Curve

As will be appreciated by those skilled in the art, the exact nature of the plasma concentration curve will be influenced by the combination of all of these factors just described. In particular, the lag time between the delivery (and thus also the onset of action) of the acylanilide, and preferably bicalutamide, in each component may be controlled by varying the bicalutamide, and coating (if present) of each of the components. Thus, by variation of each component (including the amount and nature of the bicalutamide and by variation of the lag time, numerous release and plasma profiles may be obtained. Depending on the duration of the lag time between the release of bicalutamide from each component and the nature of the release constituent (i.e., immediate release, sustained release etc.), the pulses in the plasma profile may be well separated and clearly defined peaks (e.g., when the lag time is long) or the pulses may be superimposed to a degree (e.g. in when the lag time is short).

In a preferred embodiment, the controlled release composition according to the present invention has a first immediate release component and at least one subsequent or modified release component. The immediate release component comprises a first population of active ingredient-containing particles, preferably bicalutamide nanoparticles, and the modified release components or formulations comprise second and subsequent populations of active ingredient-containing particles, preferably bicalutamide nanoparticles. The second and subsequent modified release components or formulations may comprise a modified release coating. Additionally or alternatively, the second and subsequent modified release components or formulations may comprise a modified release matrix material. In operation, administration of such a modified release component or formulation having, for example, a single modified release constituent, results in characteristic pulsatile plasma concentration levels of the bicalutamide in which the immediate release constituent of the composition gives rise to a first peak in the plasma profile and the modified release constituent gives rise to a second peak in the plasma profile. Embodiments of the invention comprising more than one modified release constituent give rise to further peaks in the plasma profile.

Such a plasma profile produced from the administration of a single dosage unit is advantageous when it is desirable to deliver two (or more) pulses of active ingredient without the need for administration of two (or more) dosage units. Additionally, in particular, in combination therapy with a luteinizing hormone-release hormone (LHRH) analogue for the treatment of stage D₂ metastatic carcinoma of the prostate, it is particularly useful to have such a bimodal plasma profile. For example, a typical bicalutamide treatment regime consists of administration of two doses of an immediate release dosage formulation given four hours apart. This type of regime has been found to be therapeutically effective and is widely used. As previously mentioned, the development of patient tolerance is an adverse effect sometimes associated with bicalutamide treatments. It is believed that the trough in the plasma profile between the two peak plasma concentrations is advantageous in reducing the development of patient tolerance by providing a period of wash-out of the bicalutamide. Drug delivery systems which provide zero order or pseudo zero order delivery of the bicalutamide do not facilitate this wash-out process.

Modified Release Coating Material

Any coating material which modifies the release of the acylanilide, and preferably bicalutamide, in the desired manner may be used. In particular, coating materials suitable for use in the practice of the invention include but are not limited to polymer coating materials, such as cellulose acetate phthalate, cellulose acetate trimaletate, hydroxy propyl methylcellulose phthalate, polyvinyl acetate phthalate, ammonio methacrylate copolymers such as those sold under the Trade Mark Eudragit.RTM. RS and RL, poly acrylic acid and poly acrylate and methacrylate copolymers such as those sold under the Trade Mark Eudragite S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, gelatin, starch, and cellulose based cross-linked polymers—in which the degree of crosslinking is low so as to facilitate adsorption of water and expansion of the polymer matrix, hydoxypropyl cellulose, hydroxypropyl methylcellulose, polyvinylpyrrolidone, crosslinked starch, microcrystalline cellulose, chitin, aminoacryl-methacrylate copolymer (Eudragit.RTM. RS-PM, Rohm & Haas), pullulan, collagen, casein, agar, gum arabic, sodium carboxymethyl cellulose, (swellable hydrophilic polymers) poly(hydroxyalkyl methacrylate) (m. wt. .about.5 k-5,000 k), polyvinylpyrrolidone (m. wt. .about.10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. .about.30 k-300 k), polysaccharides such as agar, acacia, karaya, tragacanth, algins and guar, polyacrylamides, Polyox.RTM. polyethylene oxides (m. wt. .about.100 k-5,000 k), AquaKeep.RTM. acrylate polymers, diesters of polyglucan, crosslinked polyvinyl alcohol and poly N-vinyl-2-pyrrolidone, sodium starch glucolate (e.g., Explotab.RTM.; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g., Polyox.RTM., Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g., Eudragit.RTM., Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof. As will be appreciated by the person skilled in the art, excipients such as plasticizers, lubricants, solvents and the like may be added to the coating. Suitable plasticizers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidized tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

Modified Release Matrix Material

When the subsequent component or formulation comprises a modified release matrix material, any suitable modified release matrix material or suitable combination of modified release matrix materials may be used. Such materials are known to those skilled in the art. The term “modified release matrix material” as used herein includes hydrophilic polymers, hydrophobic polymers and mixtures thereof which are capable of modifying the release of the acylanilide, and preferably bicalutamide, dispersed therein in vitro or in vivo. Modified release matrix materials suitable for the practice of the present invention include but are not limited to microcrystalline cellulose, sodium carboxymethyl-cellulose, hydoxyalkylcelluloses such as hydroxypropylmethylcellulose and hydroxypropylcellulose, polyethylene oxide, alkylcelluloses such as methylcellulose and ethylcellulose, polyethylene glycol, polyvinylpyrrolidone, cellulose acteate, cellulose acetate butyrate, cellulose acteate phthalate, cellulose acteate trimellitate, polyvinylacetate phthalate, polyalkylmethacrylates, polyvinyl acetate and mixture thereof.

Form of Dosage

A controlled release composition according to the present invention may be incorporated into any suitable dosage form which facilitates release of the active ingredient in a pulsatile manner. Typically, the dosage form may be a blend of the different populations of bicalutamide nanoparticles, in particular, in combination therapy with a luteinizing hormone-releasing hormone (LHRH) analogue for the treatment of stage D₂ metastatic carcinoma of the prostate. The bicalutamide-containing particles, preferably nanoparticles, which make up the immediate release and the modified release components, may be blended, and the blend filled into suitable capsules, such as hard or soft gelatin capsules. Alternatively, the different individual populations of active ingredient-containing particles may be compressed (optionally with additional excipients) into mini-tablets which may be subsequently filled into capsules in the appropriate proportions. Another suitable dosage form is that of a multilayer tablet. In this instance the first component of the controlled release composition may be compressed into one layer, with the second component or formulation being subsequently added as a second layer of the multilayer tablet. The populations of acylanilide, and preferably bicalutamide-containing particles, preferably nanoparticles, making up the composition of the invention may further be included in rapidly dissolving dosage forms such as an effervescent dosage form or a fast-melt dosage form.

The composition according to the invention preferably comprises at least two populations of bicalutamide nanoparticles which have different in vitro dissolution profiles.

Preferably, in operation, the composition of the invention and the solid oral dosage forms containing the composition release the acylanilide, and preferably bicalutamide, in a manner that substantially all of the acylanilide, and preferably bicalutamide, contained in the first component is released prior to release of the acylanilide, and preferably bicalutamide, from the second component. When the first component comprises an IR component, for example, it is preferable that release of the bicalutamide from the second or subsequent component is delayed until substantially all the bicalutamide in the IR component has been released. Release of the bicalutamide from the second component may be delayed as detailed above by the use of a modified release coating and/or a modified release matrix material as part of erodable, diffusion controlled or osmotic controlled formulations.

More preferably, when it is desirable to minimize patient tolerance by providing a dosage regime which facilitates wash-out of a first dose of bicalutamide from a patient's system, release of the bicalutamide from the second component or formulation is delayed until substantially all of the bicalutamide contained in the first component has been released, and further delayed until at least a portion of the bicalutamide released from the first component has been cleared from the patient's system. In a preferred embodiment, release of the bicalutamide from the second component of the composition in operation is substantially, if not completely, delayed for a period of at least about two hours after administration of the composition and is released preferably over the remaining twenty-four hour period after administration.

It will be apparent to those skilled in the art that various modifications and variations can be made in the methods and compositions of the present invention without departing from the spirit or scope of the invention. Thus, it is intended that the present invention cover the modifications and variations of this invention, provided they come within the scope of the appended claims and their equivalents.

We claim:

1. A controlled release composition consisting essentially of a first component comprising a first population of acylanilide particles and at least one subsequent component or formulation comprising a subsequent population of acylanilide particles and a modified release constituent comprising a modified release coating, a modified release matrix material or mixtures thereof, wherein the composition following oral delivery to a subject, delivers the acylanilide in the first and subsequent populations in a pulsatile manner.

2. The composition of claim 1, wherein the acylanilide in the first and subsequent populations is bicalutamide and said modified release constituent delivers to a subject the subsequent population of bicalutamide over a period of up to twenty-four hours after administration.

3. The composition according to claim 2, wherein the first and subsequent components comprise bicalutamide nanoparticles.

4. The composition according to claim 3, wherein said nanoparticles have an effective average particle size of less than 2000 nm.

5. The composition according to claim 1, wherein the first population comprises an immediate-release constituent and the formulation comprising the subsequent population is an erodable formulation.

6. The composition according to claim 1, wherein the formulation comprising the subsequent population is a diffusion controlled formulation.

7. The composition according to claim 1, wherein the formulation comprising the subsequent population is an osmotic controlled formulation.

8. The compositions of claim 3, wherein the formulation comprises a modified release coating.

9. The composition according to claim 8, wherein the composition further comprises an enhancer.

10. The composition according to claim 9, wherein the amount of bicalutamide contained in each of the first and subsequent populations is from about 0.1 mg to about 50 mg.

11. The composition according to claim 10, wherein the first and subsequent populations have different in vitro dissolution profiles.

12. The composition according to claim 11, which in operation releases substantially all of the bicalutamide from the first population prior to release of the bicalutamide from the subsequent population.

13. The composition according to claim 12 comprising a blend of the bicalutamide nanoparticles of each of the first and subsequent populations contained in a hard gelatin or soft gelatin capsule.

14. The composition according to claim 2, wherein the bicalutamide particles of each of the populations are in the form of mini-tablets and the capsule contains a mixture of the mini-tablets.

15. The composition according to claim 2, in the form of a multilayer tablet comprising a first layer of compressed bicalutamide particles of the first population and another layer of compressed bicalutamide particles and a second active ingredient-containing particles of the subsequent population.

16. The composition according to claim 15, wherein the first and subsequent populations of bicalutamide-containing particles are provided in a rapidly dissolving dosage form.

17. The composition according to claim 16, wherein the particles of each of the populations are compressed into a fast-melt tablet.

18. A method for the treatment of stage D₂ metastatic carcinoma of the prostate, comprising a combination therapy, wherein a therapeutically effective amount of a composition according to claim 2 is administered with a luteinizing hormone-release hormone (LHRH) analogue, to a patient in need thereof.

19. The composition according to claim 2, wherein the subsequent formulation comprises a pH-dependent polymer coating which is effective in releasing a pulse of the active ingredient following a time delay.

20. The composition according to claim 19, wherein the polymer coating comprises methacrylate copolymers.

21. The composition according to claim 20, wherein the polymer coating comprises a mixture of methacrylate and ammonio methacrylate copolymers in a ratio sufficient to achieve a pulse of the active ingredient following a time delay.

22. The composition according to claim 21, wherein the ratio of methacrylate to ammonio methacrylate copolymers is 1:1.
